(19) United States
(12) Patent Application Publication
(10) Pub. No.: US 2009/0088837 A1
(43) Pub. Date: Apr. 2, 2009

(54) PROSTHETIC CHORDAE ASSEMBLY AND METHOD OF USE

(75) Inventors: A. Marc Gillinov, Orange Village, OH (US); Michael K. Banbury, Rockland, DE (US)

Correspondence Address: TAROLLI, SUNDHEIM, COVELL & TUMMINO L.L.P., 1300 EAST NINTH STREET, SUITE 1700, CLEVEVLAND, OH, 44114, US

(73) Assignee: The Cleveland Clinic Foundation

(21) Appl. No.: 12/238,322

(22) Filed: Sep. 25, 2008

Related U.S. Application Data
(60) Provisional application No. 60/975,886, filed on Sep. 28, 2007.

(51) Int. Cl. A61F 2/24; A61B 19/00; A61B 17/04; A61B 5/00

(52) U.S. Cl. 623/2.1; 128/898; 606/228; 600/301

(57) ABSTRACT

A prosthetic chordae assembly includes a plurality of equally-sized, interconnected loop members formed from a single strand of a biocompatible material. Each of the loop members includes first and second ends respectively defining a first arcuate junction and a second arcuate junction. The prosthetic chordae assembly is adjustable to a pre-determined length by removing one of the loop members.

### RELATED APPLICATION

This application claims priority from U.S. provisional patent application Ser. No. 60/975,886, filed Sep. 28, 2007, the entirety of which is hereby incorporated by reference.

### TECHNICAL FIELD

The present invention generally relates to artificial chordae, and more particularly to a prosthetic chordae assembly for mitral or tricuspid valve repair.

### BACKGROUND OF THE INVENTION

Heart valve replacement is a well known procedure in which an artificial heart valve prosthesis is implanted in place of a diseased or malfunctioning heart valve. Heart valve prostheses may be mechanical or bioprosthetic. Use of mechanical valves typically requires extensive anticoagulation therapy. The need for anticoagulation therapy can be avoided in general by the use of artificial biological heart valves, such as bovine xenografts. Nevertheless, dystrophic calcification with subsequent degeneration is the major cause of failure of such bioprostheses in the long term.

When mitral or tricuspid valve replacement is performed, the chordae tendineae are cut, thus leaving the geometry and function of the ventricle impaired and in need of reconstruction. As an alternative to conventional heart valve replacement operations, diseased and malfunctioning chordae can be repaired by surgically replacing diseased heart chordae with artificial chordae. One known way of replacing a malfunctioning chordae uses a simple suture with a needle on each end of the suture. The suture is stitched through the papillary muscle and secured thereto with a knot. The two ends of the suture are then similarly stitched through the free ends of the valve leaflets.

Operations to repair heart valve chordae are technically demanding. For exam pie, when a second knot is needed to secure the suture to the valve leaflets, the length of the suture spanning the distance between the papillary muscle and the valve leaflet is likely to change since there is nothing holding the suture in place. This complication increases the skill and time required to perform the procedure. Moreover, the valve will not function properly if the length of the artificial chordae between the papillary muscle and valve leaflet is overly long or overly short.

### SUMMARY OF THE INVENTION

According to one aspect of the present invention, a prosthetic chordae assembly comprises a plurality of equally-sized, interconnected loop members formed from a single strand of a biocompatible material. Each of the loop members includes first and second ends respectively defining a first arcuate junction and a second arcuate junction. The prosthetic chordae assembly is adjustable to a pre-determined length by removing one of the loop members.

According to another aspect of the present invention, a method is provided for replacing the native chordae of a heart valve having at least two leaflets. One step of the method includes measuring the distance between a papillary muscle and a location on at least one of the at least two leaflets of the heart valve. Next, a prosthetic chordae assembly is provided. The prosthetic chordae assembly comprises a plurality of equally-sized, interconnected loop members formed from a single strand of a biocompatible material. Each of the loop members includes first and second ends respectively defining a first arcuate junction and a second arcuate junction. The length of the prosthetic chordae assembly is then adjusted to the measured distance by removing at least one of the loop members. Next, the second arcuate junction of a loop member is attached to the papillary muscle, and the first arcuate junction of another loop member is attached at the location on one of the at least two leaflets of the heart valve.

According to another aspect of the present invention, a measuring device for determining a distance between a papillary muscle and a location on a heart valve leaflet comprises a first handle member slidably connected to a second handle member. The first handle member includes a first proximal end portion and a first distal end portion. The first proximal end portion includes a first reference tab, and the first distal end portion includes a first handle. The second handle member includes a second proximal end portion and a second distal end portion. The second proximal end portion includes a second reference tab, and the second distal end portion includes a second handle. The second handle member includes a first major surface having a plurality of distance markers for indicating the distance between the papillary muscle and the location on the heart valve leaflet.

According to another aspect of the present invention, a method is provided for measuring a distance between a papillary muscle and a location on a heart valve leaflet. One step of the method includes providing a measuring device comprising a first handle member slidably connected to a second handle member. The first handle member includes a first proximal end portion with a reference tab and a first distal end portion with a first handle. The second handle member includes a second proximal end portion with a second reference tab and a second distal end portion with a second handle. The second handle member also includes a first major surface having a plurality of distance markers. The measuring device is inserted into a cardiac ventricle. The second proximal end portion of the second handle member is then positioned substantially adjacent the papillary muscle. Next, the first handle member is manipulated so that the first proximal end portion of the first handle member is substantially adjacent the location on the heart valve leaflet. The distance between the first and second reference tabs is then determined.

According to another aspect of the present invention, a prosthetic chordae assembly comprises an elongated, belt-like strap member having a first major surface oppositely disposed from a second major surface. The strap member includes a plurality of apertures extending between the first and second major surfaces.

### BRIEF DESCRIPTION OF THE DRAWINGS

The foregoing and other features of the present invention will become apparent to those skied in the art to which the present invention relates upon reading the following description with reference to the accompanying drawings, in which:

FIG. 1 is a plan view of an apparatus for replacing the native chordae of a heart valve having at least two leaflets constructed in accordance with the present invention;

FIG. 2 is a cross-sectional view of a human heart;

FIG. 3 is a plan view showing an alternative embodiment of the apparatus in FIG. 1;

FIG. 4 is an exploded cross-sectional view of the human heart in FIG. 2 showing a regurgitant mitral valve;

FIG. 5A is a perspective view showing a measuring device comprising first and second handle members for determining the distance between a papillary muscle and a location on a heart valve leaflet;

FIG. 5B is a perspective view of the first handle member;

FIG. 5C is a side view of the first handle member in FIG. 5B;

FIG. 5D is a cross-sectional view taken along Line 5C-5C in FIG. 5C;

FIG. 5E is a cross-sectional view showing an alternative embodiment of the first handle member in FIG. 5D;

FIG. 5F is a perspective view of the second handle member;

FIG. 5G is a side view of the second handle member in FIG. 5F;

FIG. 6 is a perspective view showing the measuring device in FIG. 5 being positioned between a papillary muscle and a heart valve leaflet;

FIG. 7 is a perspective view showing the measuring device in FIG. 6 being used to measure the distance between the papillary muscle and the heart valve leaflet;

FIG. 8 is a cross-sectional view showing the native chordae resected from the human heart in FIG. 4;

FIG. 9 is a top plan view showing a papillary implantation device for securing the apparatus of the present invention to a papillary muscle;

FIG. 10 is a magnified perspective view showing the papillary implantation device in FIG. 9 being used to position the apparatus in FIG. 3 about a papillary muscle;

FIG. 11 is a magnified perspective view showing the implantation device of FIG. 10 securing the apparatus in FIG. 3 to the papillary muscle;

FIG. 12 is a magnified perspective view showing the apparatus in FIG. 11 secured to the papillary muscle;

FIG. 13 is a cross-sectional view showing the apparatus in FIG. 12 secured to the papillary muscle;

FIG. 14 is a perspective view showing a leaflet implantation device for securing the apparatus of the present invention to a heart valve leaflet;

FIG. 15 is a magnified perspective view showing the leaflet implantation device in FIG. 14 being positioned about a heart valve leaflet;

FIG. 16 is a magnified perspective view showing the leaflet implantation device of FIG. 15 securing the apparatus in FIG. 3 to the heart valve leaflet;

FIG. 17 is a cross-sectional view showing the apparatus in FIG. 3 implanted in a human heart;

FIG. 18A is a perspective view showing an alternative embodiment of the apparatus in FIG. 1; and

FIG. 18B is a perspective view of the apparatus in FIG. 18A attached to a mitral leaflet and a papillary muscle.

### DETAILED DESCRIPTION

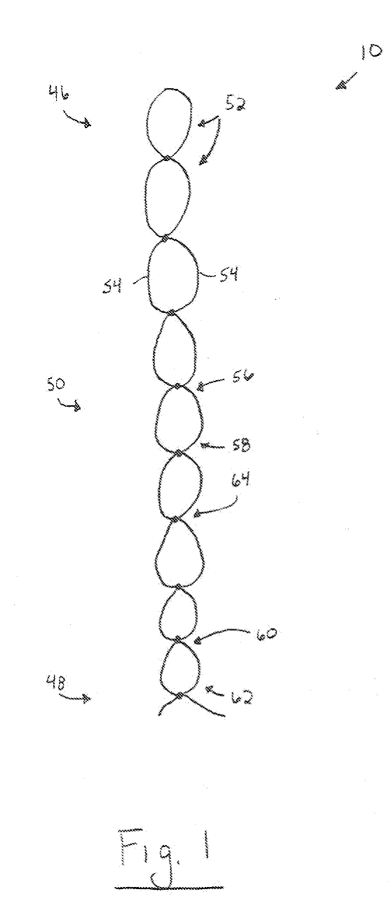

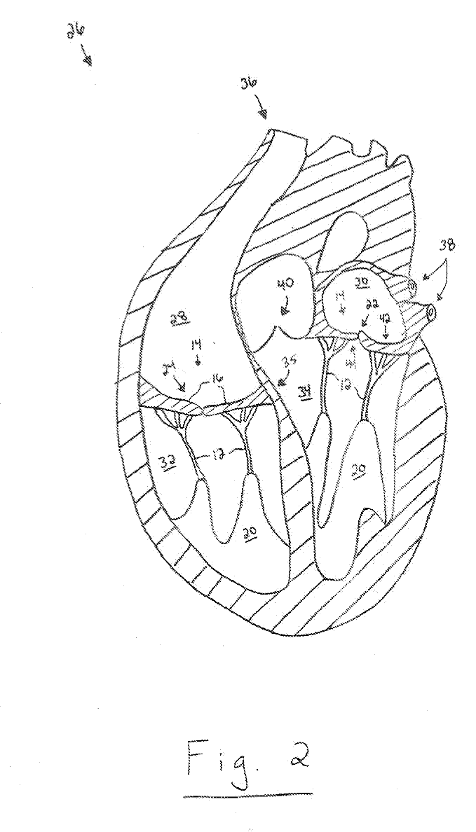

The present invention relates to artificial chordae, and more particularly to a prosthetic chordae assembly for mitral or tricuspid valve repair. As representative of the present invention, FIG. 1 illustrates an apparatus 10 for replacing the native chordae 12 (FIG. 2) of a heart valve 14 having at least two leaflets 16. The apparatus 10 (FIG. 1) comprises a prosthetic chordae assembly 18 configured to extend from a papillary muscle 20 (FIG. 2) to a location on one of the at least two heart valve leaflets 16. Although the present invention is described herein as being useful for treating a dysfunctional mitral valve 22, it should be appreciated that other cardiac valves, such as the tricuspid valve 24 are also treatable using the present invention.

FIG. 2 shows a human heart 26. The human heart 28 contains four chambers: the right and left atria 28 and 30 and the right and left ventricles 32 and 34. The thin-walled right atrium 28 receives deoxygenated blood from the superior vena cava 36, the inferior vena cava (not shown), and from the coronary sinus (not shown). The thin-wailed left atrium 30 receives oxygenated blood from pulmonary veins 38. The right and left ventricles 32 and 34 pump oxygenated and deoxygenated blood, respectively, throughout the body, and the pocket-like pulmonary (not shown) and aortic 40 semilunar valves prevent reflux into the ventricles.

Atrial blood is pumped through the atrioventricular orifices, guarded by the 3-cusp tricuspid valve 24 on the right and the 2-cusp mitral valve 22 on the left. The mitral valve 22 is formed by two leaflets; namely, the anterior leaflet 42 and the posterior leaflet 44. The anterior leaflet 42 extends along a generally planar base of a D-shaped annulus (hot shown in detail), while the posterior leaflet 44 extends arcuately around the curved portion of the annulus. The mitral and tricuspid valves 22 and 24 are secured to the papillary muscles 20 in the right and let ventricles 32 and 34 by tendinous chordae tendineae 12 and by the mitral annulus and the tricuspid annulus (not shown in detail).

Referring to FIG. 1, the prosthetic chordae assembly 18 includes a first end portion 46, a second end portion 48, and a middle portion 50 extending between the first and second end portions. The prosthetic chordae assembly 18 is comprised of a plurality of loop members 52 arranged in a daisy chain or ladder-like configuration. Each of the loop members 52 comprises two generally parallel strands 54 and includes first and second ends 56 and 58 which respectively define first and second arcuate junctions 60 and 62. The distance between the first and second arcuate junctions 60 and 62 can be about 1 cm; however, it should be appreciated that the distance between the first and second arcuate junctions will depend upon variations in subject anatomy. The two generally parallel strands 54 are fluidly connected to the first and second arcuate junctions 60 and 62 of each loop member 52. Each loop member 52 has a size and length that is substantially equal to the size and length of the other loop members. It should be appreciated, however, that each loop member 52 can have a length and size different from each of the other loop members. For example, the apparatus 10 can be configured so that prosthetic chordae assemblies 18 of different lengths can be easily constructed from a plurality of loop members 52.

Each loop member 52 is made from a biocompatible material that is relatively elastic and flexible to allow movement of the heart valve leaflets 16 during opening and closing of the valve 14. Examples of suitable biocompatible materials include Teflon and expanded polytetrafluoroethylene (ePTFE). The ePTFE may be suture material or fabric material. Besides Teflon and ePTFE, it should be apparent to one skilled in the art that there are other suitable biocompatible materials, including those which are frequently used to form sutures.

The prosthetic chordae assembly 18 shown in FIG. 1 comprises an elongated, unitary unit of nine interconnected loop members 52. The prosthetic chordae assembly 18 is comprised of a single strand or fiber, such as a suture; however, it should be appreciated that the prosthetic chordae assembly may be comprised of multiple units, e.g., multiple sutures. Where the prosthetic chordae assembly 18 is comprised of multiple sutures, for example, each of the sutures can be fixedly joined to form each of the loop members 52. It should also be appreciated that any number of loop members 52 can be used to form the prosthetic chordae assembly 18. Accordingly, the prosthetic chordae assembly 18 is adjustable to a pre-determined length by removing any desired number of loop members 52. It should be appreciated, however, that any desired number of loop members 52 may also be added to the prosthetic chordae assembly 18.

Where the prosthetic chordae assembly 18 comprises a unitary unit, each loop member 52 is interconnected at a common junction 64. The common junction 64 is formed by grouping each loop member 52 and then tying a knot to secure each loop member at the common junction. The knot may be formed by tying a portion of the prosthetic chordae assembly 18 (e.g., using an end of the suture) around the common junction 64. Alternatively, the knot may be tied by using a separate material, e.g., a separate suture. Other methods may also be used to secure each loop member 52 at the common junction 64. For instance, each loop member 52 may be secured at the common Junction 64 by gluing, stapling, pinning, or any other suitable method.

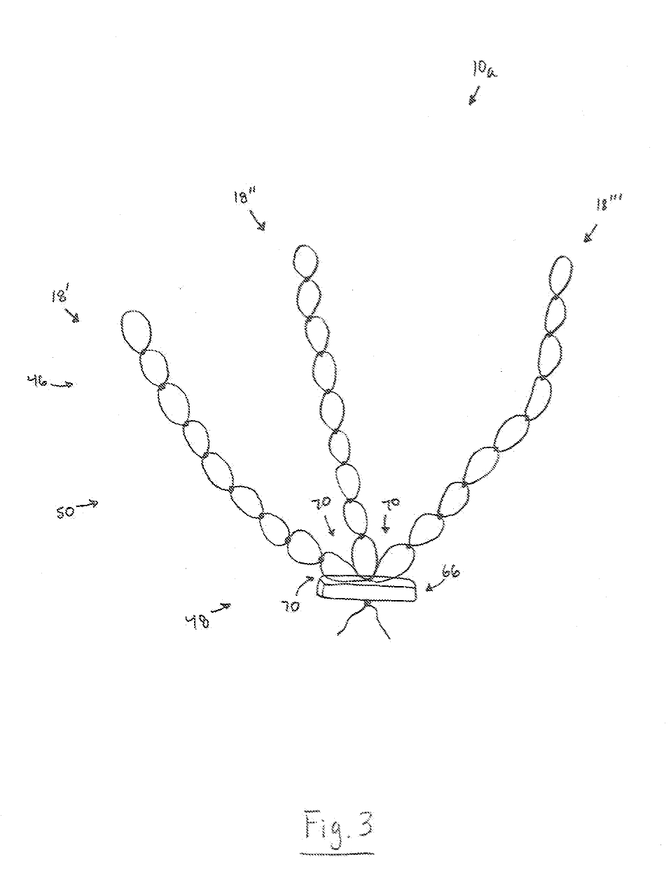

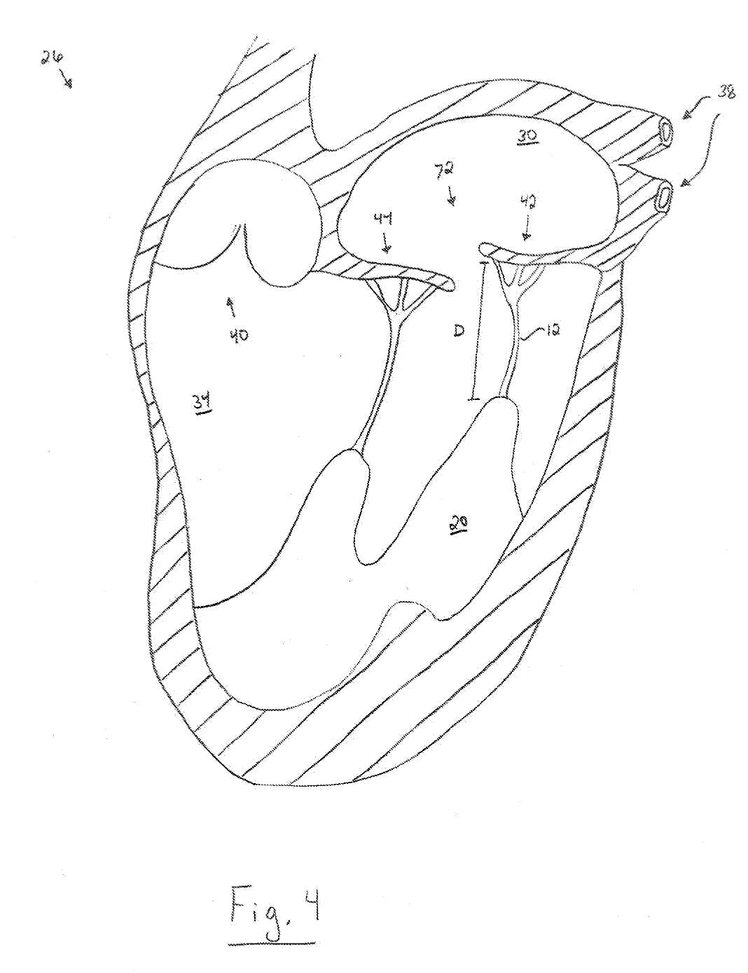

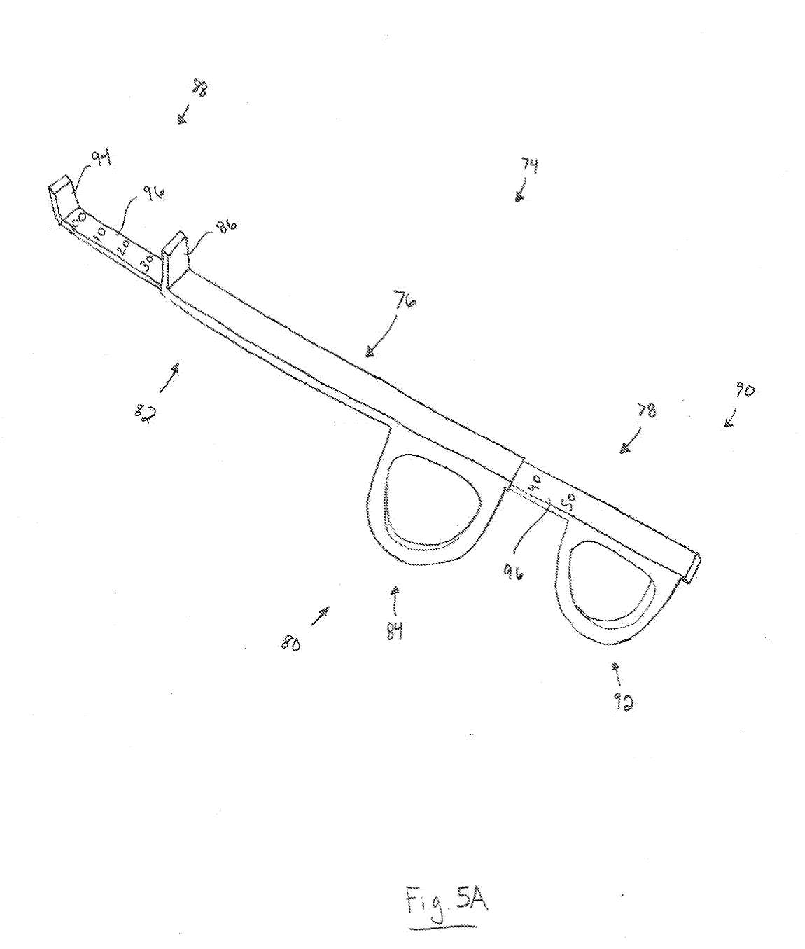

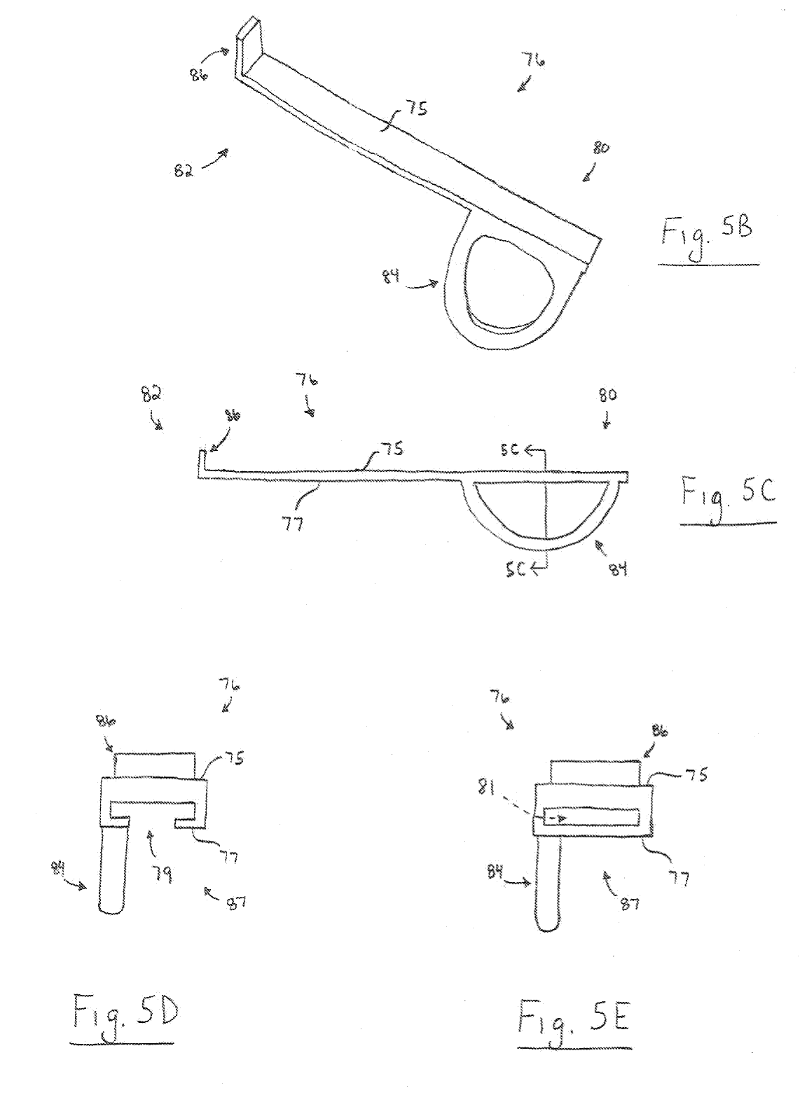

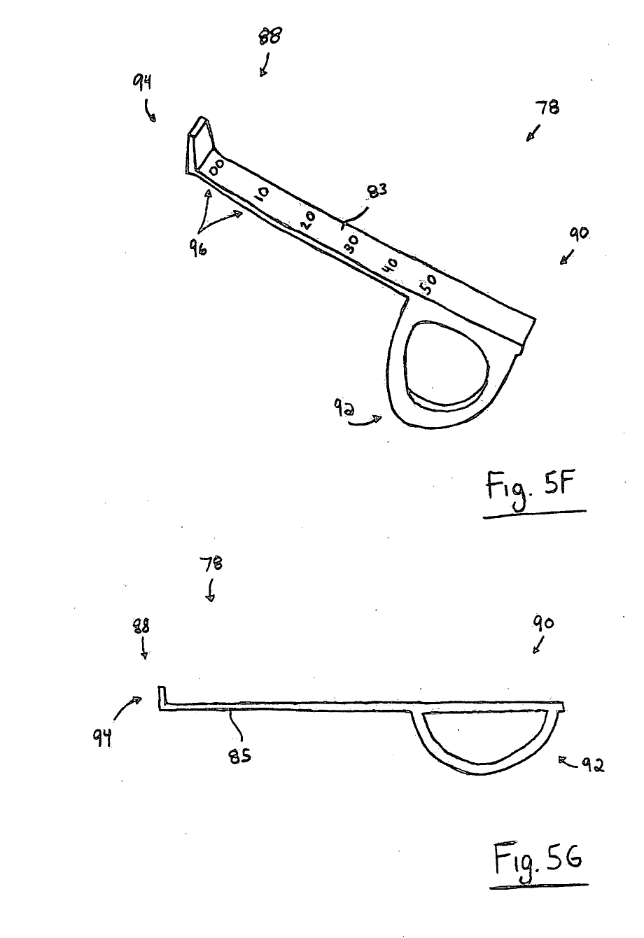

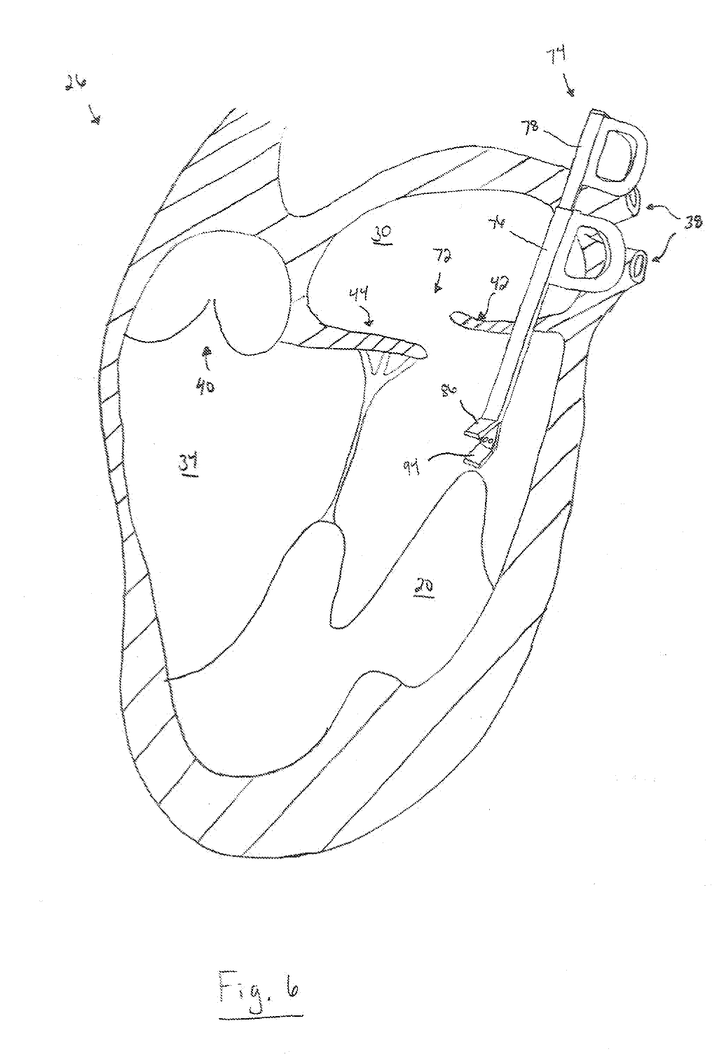

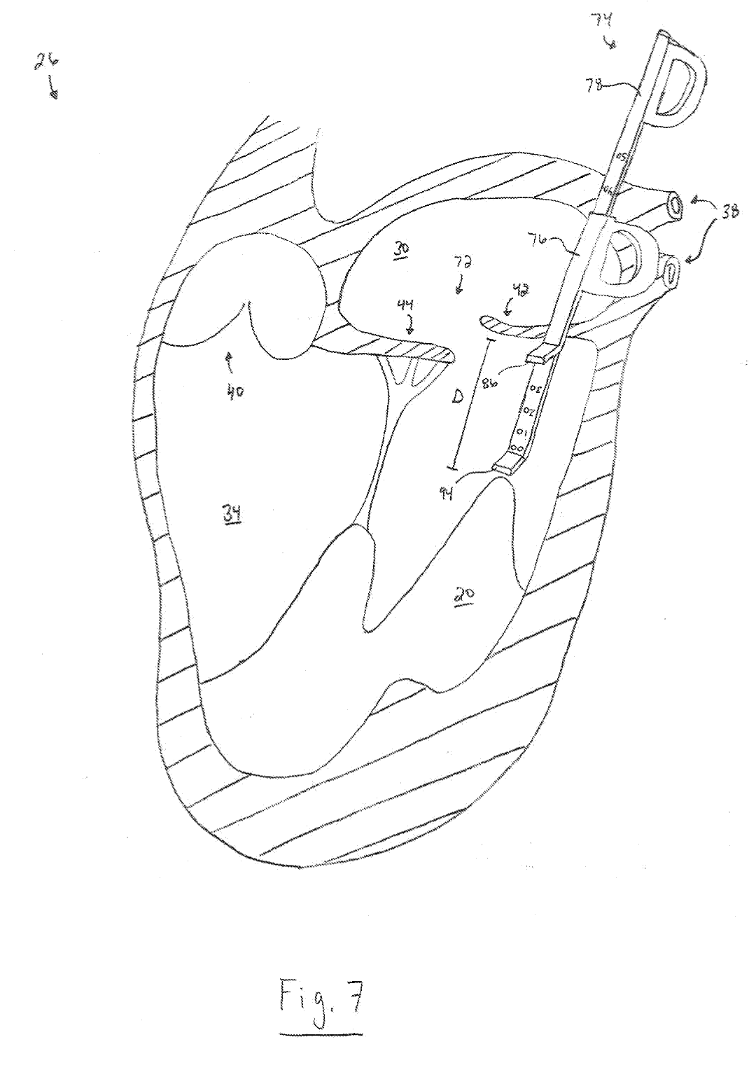

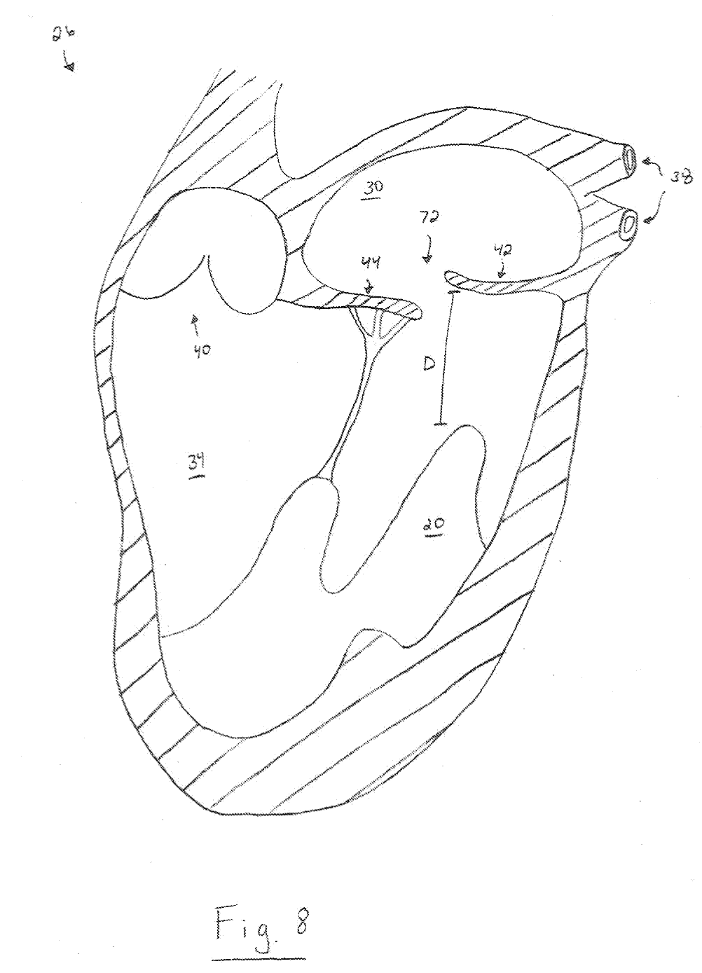

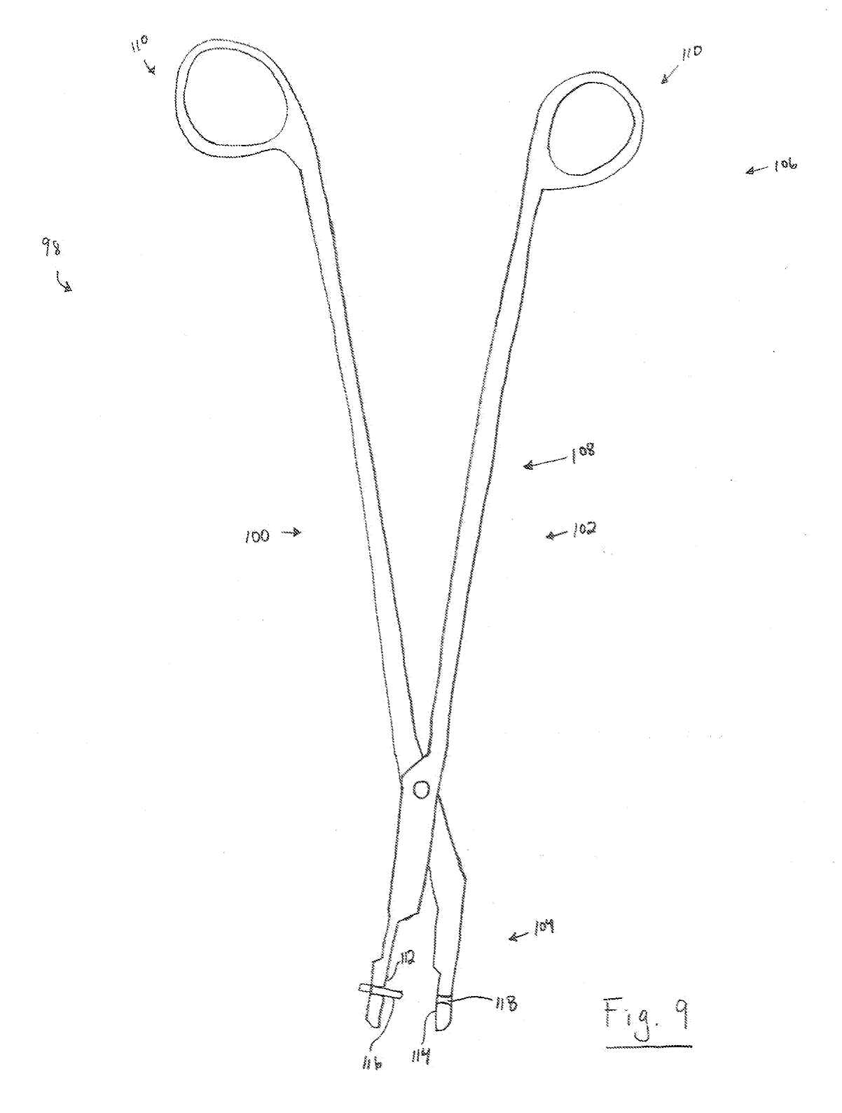

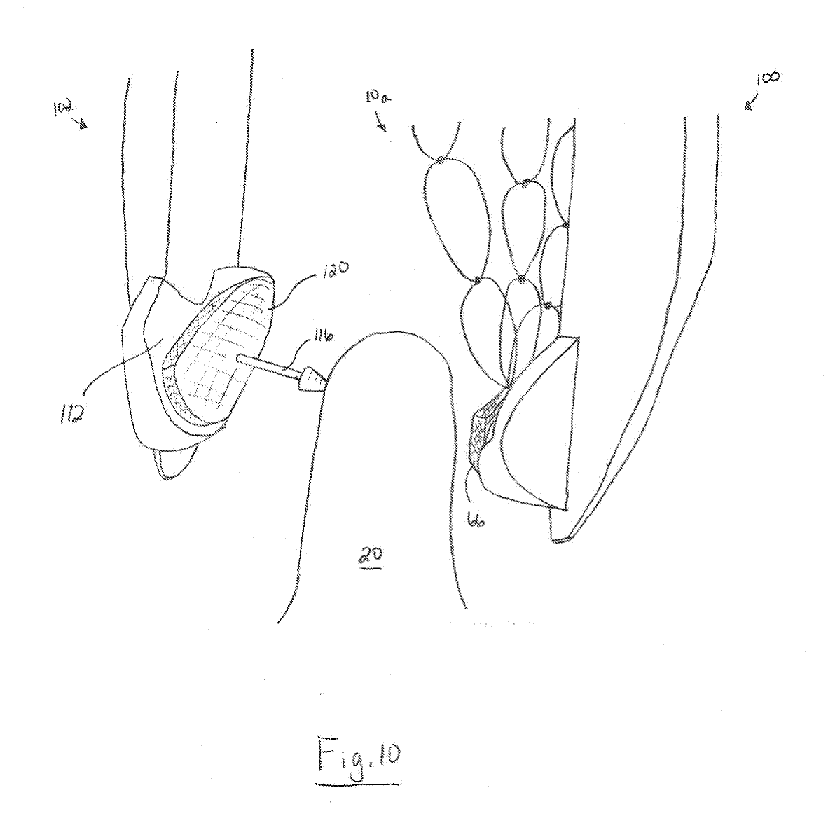

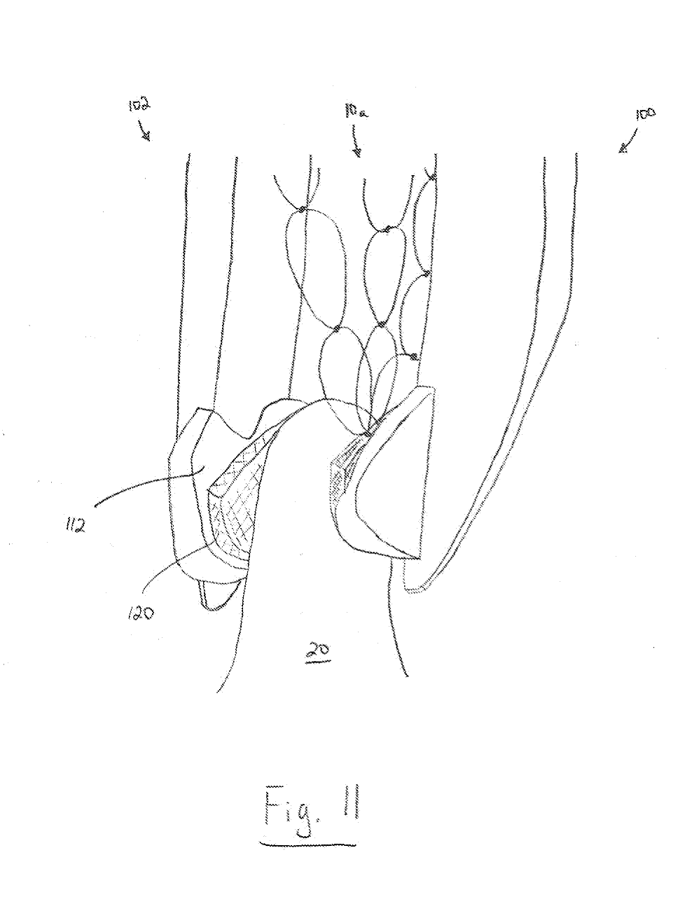

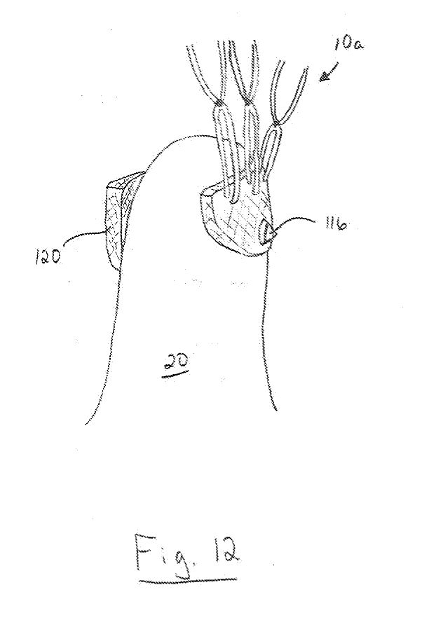

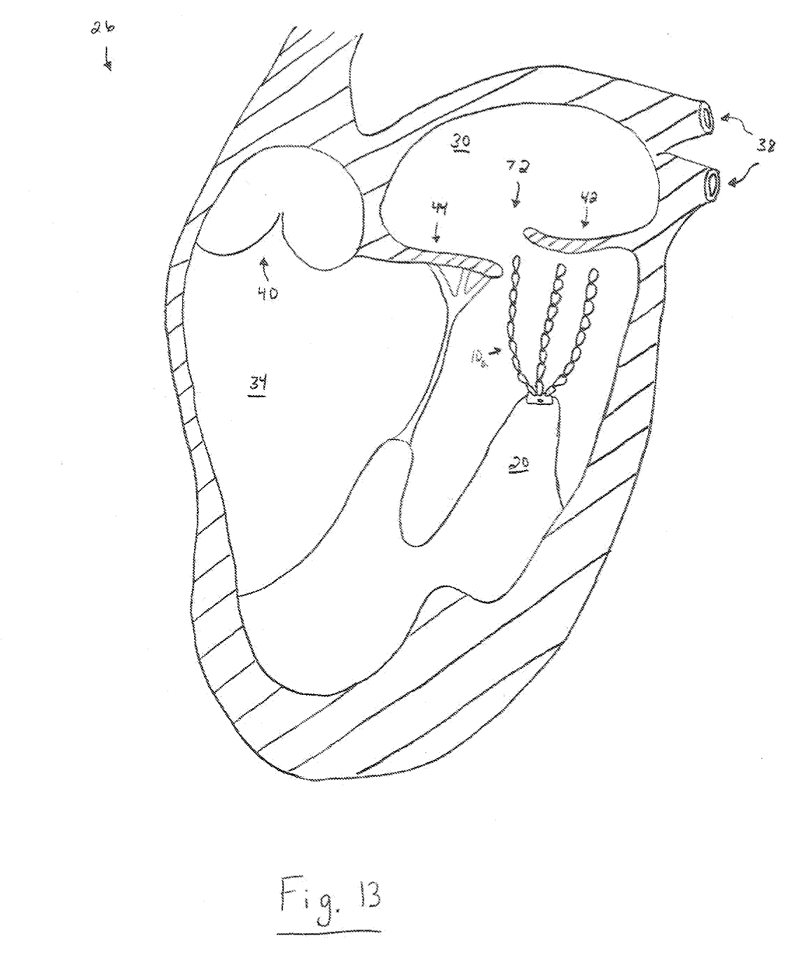

Although not shown in FIG. 1, the prosthetic chordae assembly 18 can additionally comprise a pledget 66 (FIG. 3) to facilitate attachment of the prosthetic chordae assembly to a papillary muscle 20 (FIG. 2). As shown in FIG. 3, the pledget 66 may be fixedly or slidably attached to the second end portion 48 of the prosthetic chordae assembly 18. The pledget 66 may be comprised of a non-absorbable material, such as polyurethane, silicon, polyvinyl acetate, neoprene, Teflon foam, or polyester. Alternatively, the pledget 66 may be comprised of an absorbable material, such as gelatin or collagen.

Another aspect of the present invention is illustrated in FIG. 3. The apparatus 10ₐ shown in FIG. 3 is identically constructed as the apparatus 10 shown in FIG. 1, except as described below, in FIG. 3, structures that are identical as structures in FIG. 1 use the same reference numbers, whereas structures that are similar but not identical carry the suffix “a”. The apparatus 10ₐ can be implanted during open heart surgery or, alternatively, via a catheter-based procedure.

As shown in FIG. 3, the apparatus 10ₐ can comprise a series of prosthetic chordae assemblies 18 securely attached to an attachment mechanism 68, such as a pledget 66. More particularly, the apparatus 10ₐ can include a first prosthetic chordae assembly 18′, a second prosthetic chordae assembly 18″, and a third prosthetic chordae assembly 18′″ securely attached to the pledget 66. Each of the first, second, and third prosthetic chordae assemblies 18′, 18″, and 18′″ can include an elongated, unitary unit having a first end portion 46, a second end portion 48, and a middle end portion 50 extending between the end portions. Each of the first, second, and third prosthetic chordae assemblies 18′, 18″, and 18′″ can be comprised of nine interconnected loop members 52. Each of the loop members 52 can include first and second ends 58 and 58 respectively defining first and second arcuate Junctions 60 and 62. Each loop member 52 can have a size and length that is equal to the size and length of the other loop members.

As shown in FIG. 3, the terminal loop member 70 at the second end portion 48 of each of the first, second, and third prosthetic chordae assemblies 18′, 18″, and 18′″ can be securely attached to the pledget 88. For example, the strands 54 of each terminal loop member 70 can be threaded through the pledget 66 so that each of the first, second and third prosthetic chordae assemblies 18′, 18″, and 18′″ are securely attached to the pledget. The presence of the first, second, and third prosthetic chordae assemblies 18′, 18″, and 18′″ provides multiple attachment points for securing the first arcuate junction 60 of each of the terminal loop members 70 to the location on the at least one heart valve leaflet 16. It should be appreciated that the apparatus 10ₐ can include two, four, five or even more prosthetic chordae assemblies 18 depending upon, for example, the anatomy of the heart 26 and the medical condition being treated.

The replacement of native chordae 12 of a human heart 26 with the present invention is illustrated in FIGS. 4-17. As shown in FIGS. 4-17, the apparatus 10ₐ of FIG. 3 is used to replace the native chordae 12 of a regurgitant mitral valve 72. It should be appreciated that more than one apparatus 10ₐ may be used, i.e., to replace the other set of native chordae 12. Additionally, it should be appreciated that the apparatus 10ₐ can be fixed to a portion of the left ventricular wail, the right ventricular wall, or the septum 35 (FIG. 2).

To replace the native chordae 12, the distance D (FIG. 4) between a papillary muscle 20 and a location on the anterior mitral valve leaflet 42 is first measured. Access to the native chordae 12 may be obtained by open-heart surgery, for example. During the surgery, the native chordae 12 are inspected to determine the size of the apparatus 10ₐ needed to replace the native chordae. Generally, the size of the apparatus 10ₐ needed will depend on the size of the heart 26 as well as the appropriate implantation site.

A measuring device 74, such as the one shown in FIGS. 5A-G is then used to measure a distance D between the papillary muscle 20 and the location on the anterior mitral valve leaflet 42. As shown in FIG. 5A, the measuring device 74 comprises a first handle member 76 slidably attached to a second handle member 78. The first handle member 76 (FIGS. 5B-C) includes a first distal end portion 80 and first proximal end portion 82. Additionally, the first handle member 76 includes oppositely disposed first and second major surfaces 75 and 77. The first and second handle members 76 and 78 can be made from one or a combination of known medical grade materials, such as a hardened plastic and/or metal alloy.

The first distal end portion 80 of the first handle member 76 includes a handle 84 to manipulate the measuring device 74 and facilitate longitudinal movement of the first handle member relative to the second handle member 78. The first proximal end portion 82 of the first handle member 76 includes a first reference tab 88 that extends in a substantially radial direction from the first major surface 75. The first reference tab 86 can have a rigid, semi-rigid, or flexible configuration.

The first handle member 76 also includes a sliding mechanism 87, such as a channel 79 (FIG. 5D) or a lumen 81 (FIG. 5E) for slidably receiving the second handle member 78. The channel 79 or lumen 81 can extend between the first proximal end portion 82 and the first distal end portion 80 of the first handle member 78. As shown in FIGS. 5D-E, the cross-sectional shape of the channel 79 or lumen 81 corresponds to the cross-sectional shape of the second handle member 78. Depending upon the configuration of the sliding mechanism 87, the position of the first handle 84 about the second major surface 77 can be varied as needed.

The second handle member 78 (FIGS. 5F-G) includes a second proximal end portion 88 and a second distal end portion 90. Additionally, the second handle member 78 includes a first major surface 83 oppositely disposed from a second major surface 85. The second distal end portion 90 includes a second handle 92 for holding the measuring device 74 and manipulating the second handle member 78 relative to the first handle member 78. The second proximal end portion 88 includes a second reference tab 94 that extends in a substantially radial direction from the first major surface 83. The second reference tab 94 can have a rigid, semi-rigid, or flexible configuration.

The first major surface 83 of the second handle member 78 also includes a plurality of distance markers 96. The distance markers 96 include a series of spaced apart indicia that may be used to determine the distance D between a papillary muscle 20 and a location on the anterior mitral valve leaflet 42. As shown in FIGS. 5A and 5G, for example, the distance markers 96 include a series of numerical values ranging from 0 mm to 50 mm. It will be appreciated that any range of values may be used for the distance markers 96. Additionally, it should be appreciated that other devices for measuring the distance D between the papillary muscle 20 and a location on at least one heart valve leaflet 16, such as the devices disclosed in U.S. Patent App. No. 2003/0105519A1 are known in the art and may alternatively be used for measuring the distance D between the papillary muscle and a location on at least one heart valve leaflet.

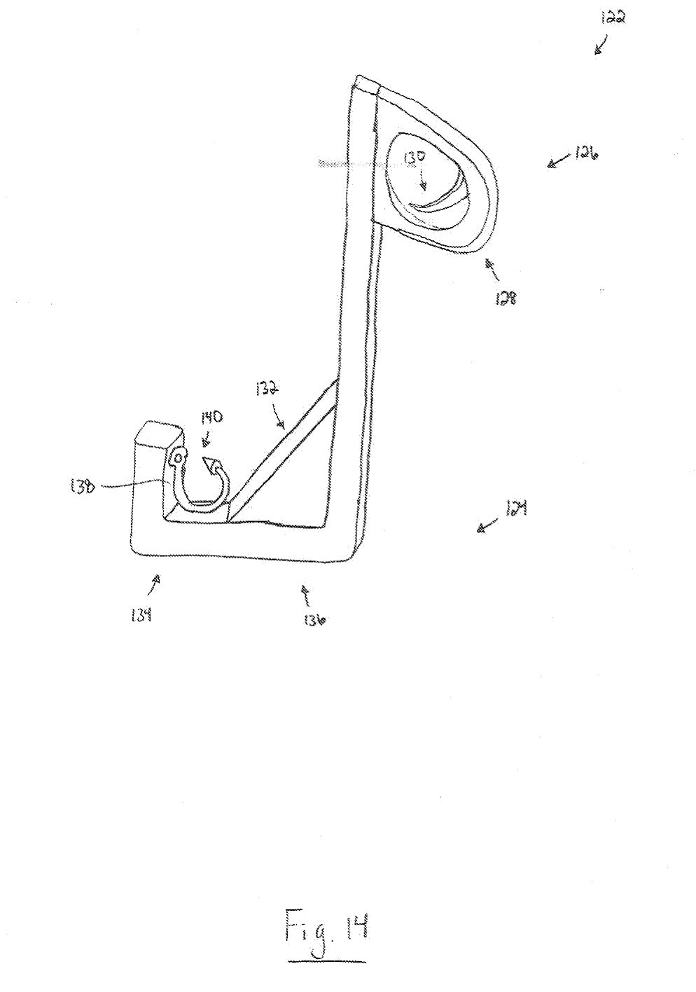

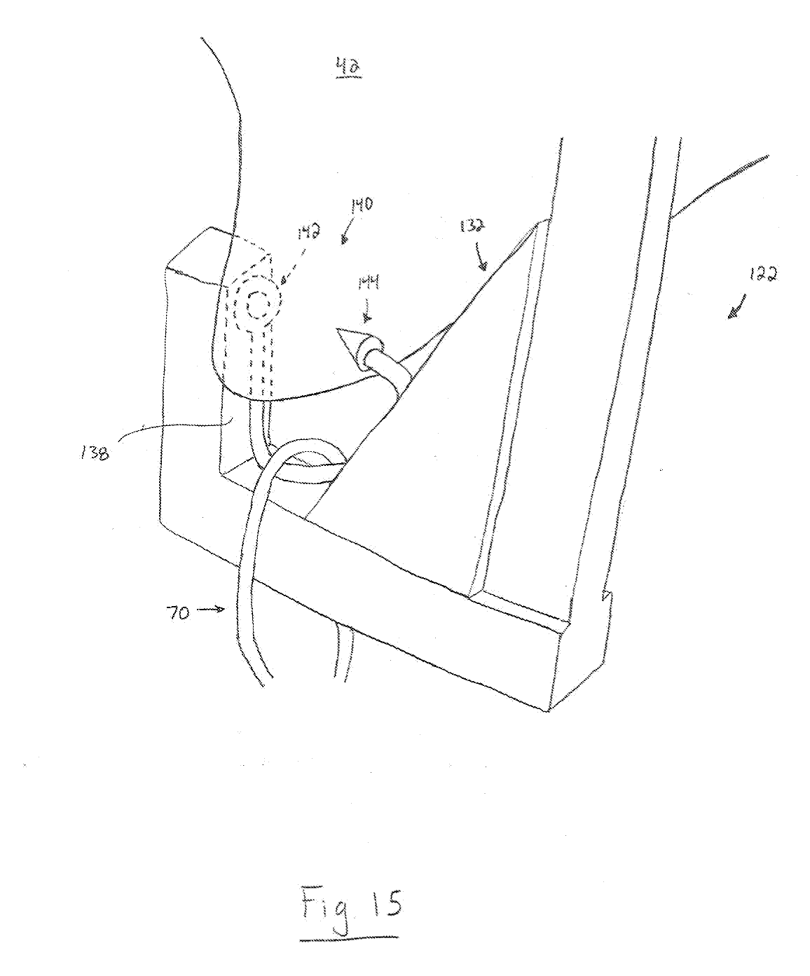

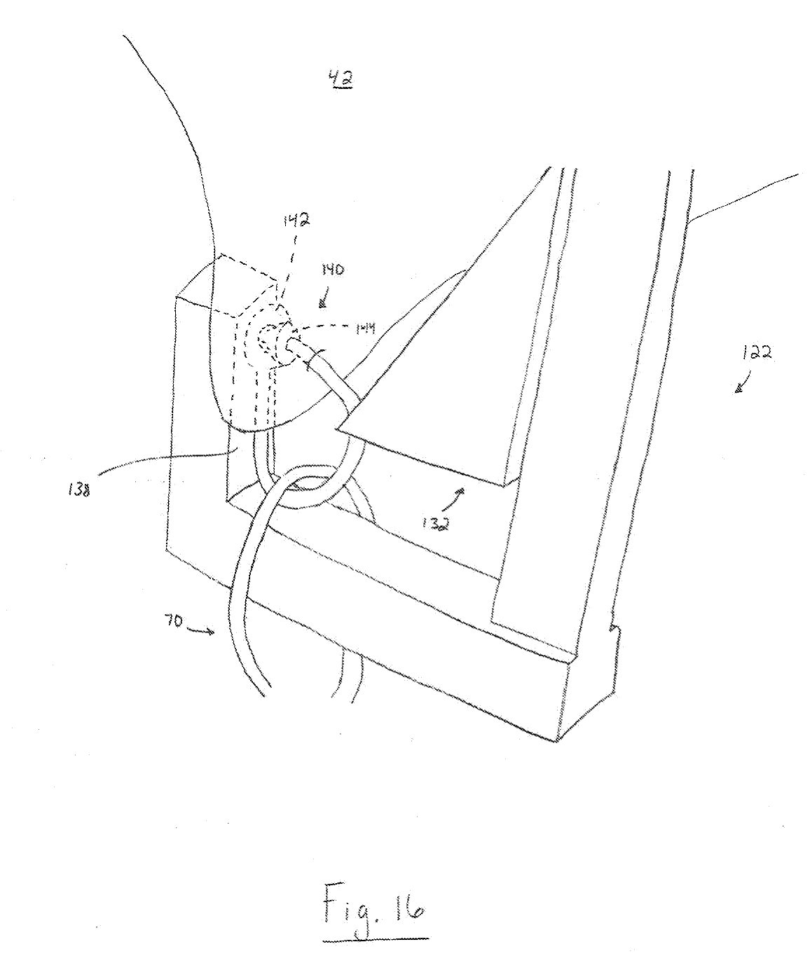

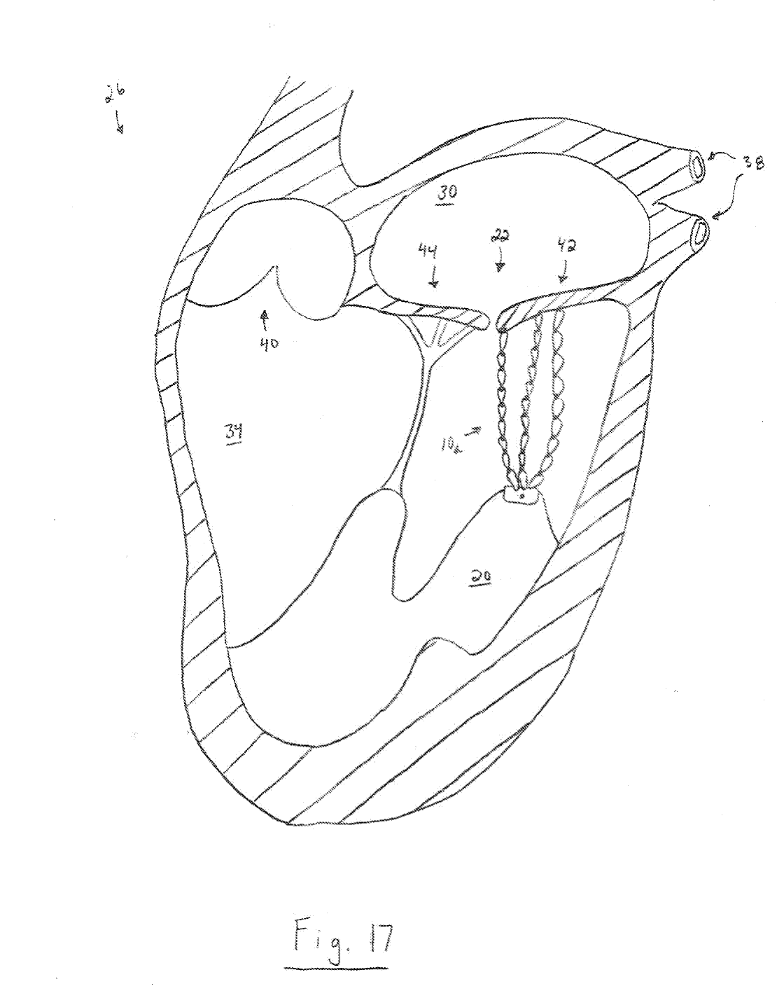

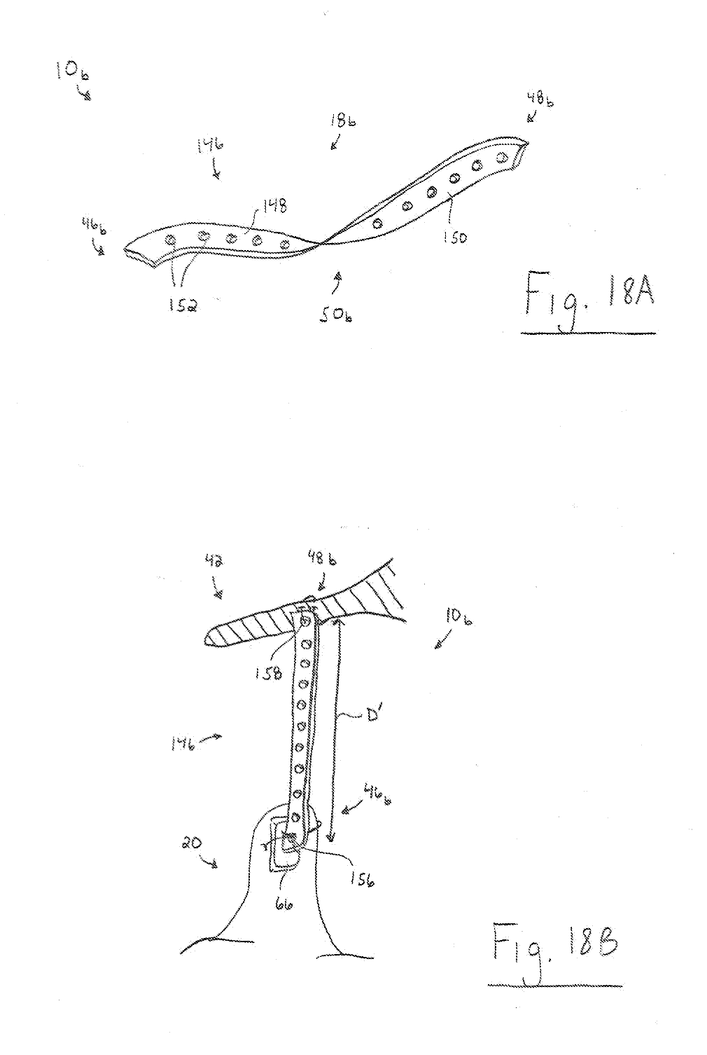

As shown in FIG. 6, the measuring device 74 is positioned in the left ventricle 34 of the heart 26. More particularly, the measuring device 74 is inserted into the left ventricle 34 so that the first and second proximal end portions 82 and 88 of each of the first and second handle members 76 and 78 are substantially adjacent the papillary muscle 20 (e.g., the apical surface of the papillary muscle), and the first reference tab 86 is positioned at the distance marker 96 corresponding to 0 mm. The first handle member 76 is then moved distally relative to the second handle member 78 until the first reference tab 86 is positioned substantially adjacent the location on the anterior mitral valve leaflet 42. The measuring device 74 is held in place so that the numerical value corresponding to the position of the first reference tab 86 can be determined. As shown in FIG. 7, the numerical value corresponding to the first reference tab 86 is approximately 40 mm. After determining the distance D between the papillary muscle 20 and the location on the anterior mitral valve leaflet 42 (i.e., 40 mm), the native chordae 12 are then resected as shown in FIG. 8.

After measuring the distance D between the papillary muscle 20 and the location on the anterior mitral valve leaflet 42, an appropriately-sized apparatus 10ₐ is selected. The size of the loop members 52 comprising each of the first, second, and third prosthetic chordae assemblies 18′, 18″, and 18′″ defines the size and length of the implanted apparatus 10ₐ. Accordingly, an apparatus 10ₐ having first, second, and third prosthetic chordae assemblies 18′, 18″, and 18′″, each of which has a pre-determined size equal to the measured distance D between the papillary muscle 20 and the location on the anterior mitral valve leaflet 42, is selected for implantation. Failure to accurately determine the correct size of the apparatus 10ₐ may result in an ineffective repair and, in turn, cause prolapse of the anterior mitral valve leaflet 42.

Once an appropriately-sized apparatus 10ₐ has been selected, the apparatus is attached to the papillary muscle 20 using a papillary implantation device 98 (FIG. 9). As shown in FIG. 9, the papillary implantation device 98 has a scissor-like configuration and includes first and second opposing arm members 100 and 102. Each of the arm members 100 and 102 includes a proximal end portion 104, a distal end portion 106, and a middle portion 108 extending between the end portions. Each of the distal end portions 106 includes a handle 110 for operating the papillary implantation device 98. The proximal end portion 104 of the second arm member 102 has a flattened surface 112 for mating to a receiving surface 114 of the first arm member 100, and includes a holding pin 116 for securing the apparatus 10ₐ during implantation. The proximal end portion 104 of the first arm member 100 includes a channel 118 for receiving the holding pin 116.

FIGS. 10-12 illustrate attachment of the apparatus 10ₐ to the papillary muscle 20 using the papillary implantation device 98. The papillary implantation device 98 and the apparatus 10ₐ can be introduced via the outer wall of the left ventricle 34 with or without the use of a heart-lung machine (not shown). A second pledget 120 is first operably attached to the proximal end portion 104 of the second arm member 102, and the apparatus 10ₐ is then operably attached to the proximal end portion of the first arm member 100. As shown in FIG. 10, the papillary implantation device 98 is then operated so that the proximal end portion 104 of the first arm member 100 and the proximal end portion of the second arm member 102 are radially disposed about the papillary muscle 20.

After appropriately positioning the papillary implantation device 98, the papillary implantation device is operated so that the proximal end portion 104 of the first arm member 100 and the proximal end portion of the second arm member 102 engage the papillary muscle 20 as shown in FIG. 11. As the first arm member 100 is contacted with the papillary muscle 20, the holding pin 116 penetrates the papillary muscle. The papillary implantation device 98 is then continually operated until the holding pin 116 extends through the pledget 66 of the apparatus 10ₐ and thereby securely attaches the apparatus to the papillary muscle 20 (FIG. 12). The papillary implantation device 98 is then removed from the left ventricle 34, in turn leaving the apparatus 10ₐ securely attached to the papillary muscle 20 as shown in FIG. 13.

During attachment of the apparatus 10ₐ to the papillary muscle 20, it is important that undue pressure is not exerted on the papillary muscle. Excess pressure on the papillary muscle 20 may crush or deform the papillary muscle, in turn rendering the papillary muscle dysfunctional. Therefore, the papillary implantation device 98 may be operated using tactile feedback to avoid delivery of undue pressure to the papillary muscle 20. Alternatively, the papillary implantation device 98 may be used with assistance from a device (not shown) capable of preventing deformation or crushing of the papillary muscle 20.

After securing the apparatus 10ₐ to the papillary muscle 20, the terminal loop members 70 of each of the first, second, and third prosthetic chordae assemblies 18′, 18″, and 18′″ are securely attached to the location on the anterior mitral valve leaflet 42 using a leaflet implantation device 122 (FIG. 14). As shown in FIG. 14, the leaflet implantation device 122 has J-shaped configuration and includes a proximal end portion and a distal end portion 124 and 126. The distal end portion 126 includes a handle 128 with a mechanism 130 for operating the leaflet implantation device 122. As described in more detail below, the mechanism 130 can include a trigger operably linked to a crimping member 132. The proximal end portion 124 of the leaflet implantation device 122 includes a first end portion 134 and a second end portion 136. The first end portion 134 includes a receiving surface 138 for receiving a securing member 140, such as a barb or hook, and the second end portion 136 includes the crimping member 132 for attaching the securing member to the anterior mitral valve leaflet 42.

FIGS. 15-17 illustrate attachment of the terminal loop member 70 at the first end portion 46 of the first prosthetic chordae assembly 18′ to the location on the anterior mitral valve leaflet 42. As shown in FIG. 15, the first arcuate junction 60 of the terminal loop member 70 is threaded around both the securing member 140 and the first end portion 134 of the leaflet implantation device 122. The leaflet implantation device 122 is then positioned so that the location on the anterior mitral valve leaflet 42 is positioned between a receptacle portion 142 and a barb portion 144 of the securing member 140. Next, the mechanism 130 of the leaflet implantation device 122 is manipulated so that the crimping member 132 advances toward the distal end portion 126 of the leaflet implantation device. As the crimping member 132 is advanced, the crimping member engages the securing member 140 and forces the barb portion 144 through the location on the anterior mitral valve leaflet 42 and into the receptacle portion 142 (FIG. 16). The barb portion 144 then extends through the receptacle portion 142 so that the securing member 140, and thus the terminal loop member 70 of the first prosthetic chordae assembly 18′ is securely attached to the location on the anterior mitral valve leaflet 42.

After securely attaching the first prosthetic chordae assembly 18′, the same steps are repeated to securely attach the second and third prosthetic chordae assemblies 18″ and 18′″ to the anterior mitral valve leaflet 42 (FIG. 17). By including multiple prosthetic chordae assemblies 18′, 18″, and 18′″ to secure the apparatus 10ₐ at the location on the anterior mitral valve leaflet 42, the tension at any one location on the anterior mitral valve leaflet is reduced. By reducing the tension at each location, tearing or ripping of the anterior mitral valve leaflet 42 can be avoided. With the apparatus 10ₐ implanted in the left ventricle 34, the anterior mitral valve leaflet 42 is pulled back into proper alignment so that the mitral valve 22 can function properly and regurgitation through the valve is substantially reduced or prevented. Upon successful placement of the apparatus 10ₐ, effective valve 22 function is verified (i.e., proper leaflet coaptation) and the open-heart surgery is completed.

Another aspect of the present invention is shown in FIGS. 18A-B. The apparatus 10_{b} shown in FIGS. 18A-B is identically constructed as the apparatus 10 shown in FIG. 1, except as described below. In FIGS. 18A-B, structures that are identical as structures in FIG. 1 use the same reference numbers, whereas structures that are similar but not identical carry the suffix “b”.

As shown in FIGS. 18A-B, the apparatus 10_{b} can comprise a prosthetic chordae assembly 18_{b} including a first end portion 46_{b}, a second end portion 48_{b}, and a middle portion 50_{b} extending between the first and second end portions. The prosthetic chordae assembly 18_{b} can be comprised of a single, belt-like strap member 146. The strap member 146 can be made of a biocompatible material that is relatively elastic and flexible to allow movement of the heart valve leaflets 16 during opening and closing of the valve 14. Non-limiting examples of biocompatible materials that can be used to form the strap member 146 include Teflon, PTFE and ePTFE.

The strap member 146 has an elongated configuration and can include a first major surface 148 oppositely disposed from a second major surface 150. The strap member 146 can also include a plurality of apertures 152 extending between the first and second major surfaces 148 and 150. As shown in FIG. 18A, the apertures 152 can have a circular shape; however, it should be appreciated that the apertures can have any one or combination of shape's (e.g., square, rectangular, ovoid, etc.).

To implant the prosthetic chordae assembly 18_{b} in a left ventricle 34, for example, the first and second end portions 46_{b} and 48_{b} can be respectively positioned adjacent a papillary muscle 20 and a location on an anterior mitral valve leaflet 42 (FIG. 18B; entire method not shown) using the papillary implantation device 98 (as described above). It will be appreciated that the proper size of the prosthetic chordae assembly 18_{b} can be determined prior to implantation by measuring the distance D between the papillary muscle 20 and the location on the anterior mitral valve leaflet 42 (as described above). After determining the distance D, a corresponding distance D′ between a first aperture 156 and a second aperture 158 can be identified.

Next, the first end portion 46_{b} can be secured to the papillary muscle 20 by passing a suture (or other similar means) through the first aperture 156, into the pledget 66, and through the papillary muscle. The suture can then be tied into a knot to secure the first end portion 46_{b}. Additionally or optionally, it should be appreciated that a suture can be passed through the strap member 146 to secure the first end portion 46_{b} to the papillary muscle 20.

After securing the first end portion 46_{b} of the strap member 146 to the papillary muscle 20, the second end portion 48_{b} can be secured to the location on the anterior mitral valve leaflet 42 by passing a suture (or other similar means) through the second aperture 158 and into the mitral leaflet. The suture can then be tied into a knot to secure the second end portion 48_{b}. Additionally or optionally, it should be appreciated that a suture can be passed through the strap member 146 to secure the second end portion 48_{b} to the location on the anterior mitral valve leaflet 42. Upon successful placement of the prosthetic chordae assembly 18_{b}, effective valve 22 function can be verified (i.e., proper leaflet coaptation) and the surgery completed.

From the above description of the invention, those skilled in the art will perceive improvements, changes and modifications, if should be appreciated that the method of the present invention is not restricted to the order of steps presented herein. For example, the apparatus 10 may be attached to the location on at least one of the two heart valve leaflets 16 of the heart valve 14 instead of first attaching the apparatus to a papillary muscle 20. As another example, all or only a portion of the apparatus 10 may be implanted prior to excising the native chordae 12. Such improvements, changes, and modifications are within the skill of the art and are intended to be covered by the appended claims.

Having described the invention, we claim:

1. A prosthetic chordae assembly comprising:
a plurality of equally-sized, interconnected loop members formed from a single strand of a biocompatible material, each of said loop members including first and second ends respectively defining a first arcuate junction and a second arcuate junction;
wherein said prosthetic chordae assembly is adjustable to a pre-determined length by removing one of said loop members.

2. A method for replacing the native chordae of a heart valve having at least two leaflets, said method comprising the steps of:
measuring the distance between a papillary muscle and a location on at least one of the at least two leaflets of the heart valve;
providing a prosthetic chordae assembly comprising a plurality of equally-sized, interconnected loop members formed from a single strand of a biocompatible material, each of the loop members including first and second ends respectively defining a first arcuate junction and a second arcuate junction;
adjusting the length of the prosthetic chordae assembly to the measured distance by removing at least one of the loop members;
attaching the second arcuate junction of a loop member to the papillary muscle; and
attaching the first arcuate junction of another loop member at the location on one of the at least two leaflets of the heart valve.

3. A measuring device for determining a distance between a papillary muscle and a location on a heart valve leaflet, said measuring device comprising:
a first handle member including a first proximal end portion and a first distal end portion, said first proximal end portion including a first reference tab and said first distal end portion including a first handle; and
a second handle member slidably connected to said first handle member, said second handle member including a second proximal end portion and a second distal end portion, said second proximal end portion including a second reference tab and said second distal end portion including a second handle, said second handle member including a first major surface having a plurality of distance markers for indicating the distance between the papillary muscle and the location on the heart valve leaflet.

4. The measuring device of claim 3, wherein said first handle member includes a sliding mechanism for slidably receiving said second handle member and facilitating movement of said first handle member relative to said second handle member.

5. The measuring device of claim 4, wherein said sliding mechanism comprises a lumen extending between said first proximal end portion and said first distal end portion.

6. The measuring device of claim 4, wherein said sliding mechanism comprises a channel extending between said first proximal end portion and said first distal end portion.

7. The measuring device of claim 3, wherein said first and second reference tabs extend in a substantially radial direction from said first and second proximal end portions, respectively.

8. A method for measuring a distance between a papillary muscle and a location on a heart valve leaflet, said method comprising the steps of:
providing a measuring device comprising a first handle member slidably connected to a second handle member, the first handle member including a first proximal end portion with a reference tab and a first distal end portion with a first handle, the second handle member including a second proximal end portion with a second reference tab and a second distal end portion with a second handle, the second handle member also including a first major surface having a plurality of distance markers;
inserting the measuring device into a cardiac ventricle;
positioning the second proximal end portion of the second handle member substantially adjacent the papillary muscle;
manipulating the first handle member so that the first proximal end portion of the first handle member is substantially adjacent the location on the heart valve leaflet; and
determining the distance between the first and second reference tabs.

9. The method of claim 8, wherein said step of positioning the second proximal end portion of the second handle member substantially adjacent the papillary muscle further comprises positioning the second reference tab substantially adjacent an apical surface of the papillary muscle.

10. The method of claim 8, wherein said step of manipulating the first handle member further comprises positioning the first reference tab substantially adjacent the location on the heart valve leaflet.

11. The method of claim 8, wherein said step of determining the distance between the first and second reference tabs further comprises determining the position of the first reference tab with respect to at least one of the distance markers;
wherein the at least one distance marker indicates the distance between the papillary muscle and the location on the heart valve leaflet.

12. A prosthetic chordae assembly comprising:
an elongated, belt-like strap member having a first major surface oppositely disposed from a second major surface, said strap member including a plurality of apertures extending between said first and second major surfaces.
